(19) United States
(12) Patent Application Publication
(10) Pub. No.: US 2011/0020447 A1
(43) Pub. Date: Jan. 27, 2011

(54) NOVEL DOSAGE FORM

(76) Inventors: Allan James Clarke, Collegeville, PA (US); Ian Paul Conn, Essex (GB); Simon Richard Hicks, Essex (GB); Yu Li, Collegeville, PA (US); Xiaolei Wang, Essex (GB)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(21) Appl. No.: 12/528,490

(22) PCT Filed: Feb. 28, 2008
(86) PCT No.: PCT/EP08/52429

§ 371 Date: Aug. 25, 2009

(51) Int. Cl. A61K 31/55; A61K 9/30; A61K 9/44; B05D 3/00; A61P 25/00

(52) U.S. Cl. 424/467; 514/213.01; 424/475; 427/2.14

(57) ABSTRACT

The present invention relates to a novel dosage form, to a process for preparing the dosage form and to the use of the dosage form in the treatment of neurological and psychiatric disorders.

This invention relates to a novel dosage form, to a process for preparing the dosage form and to the use of the dosage form in medicine.

International Patent Application, Publication Number WO2004/056369 discloses certain benzazepine derivatives including 6-(3-cyclobutyl-2,3,4,5-tetrahydro-1H-benzo[d]azepin-7-yloxy)-N-methyl-nicotinamide.

WO2004/056369 teaches that benzazepine derivatives may be formulated using standard techniques. However, no dosage form containing 6-(3-cyclobutyl-2,3,4,5-tetrahydro-1H-benzo[d]azepin-7-yloxy)-N-methyl-nicotinamide is explicitly disclosed.

In a first aspect, the present invention provides a dosage form comprising:
a) 6-(3-cyclobutyl-2,3,4,5-tetrahydro-1H-benzo[d]azepin-7-yloxy)-N-methyl-nicotinamide or a pharmaceutically acceptable salt thereof;
b) a stabiliser, which reduces degradation of 6-(3-cyclobutyl-2,3,4,5-tetrahydro-1H-benzo[d]azepin-7-yloxy)-N-methyl-nicotinamide in the dosage form when compared to a dosage form lacking said stabiliser; and
c) a pharmaceutically acceptable excipient.

The dosage form may be adapted for administration to the patient by any desired route of administration. For example, dosage forms include those adapted for (1) oral administration such as tablets, capsules, caplets, pills, lozenges, powders, syrups, elixirs, suspensions, solutions, emulsions, sachets and cachets; (2) parenteral administration such as sterile solutions, suspensions, implants and powders for reconstitution; (3) transdermal administration such as transdermal patches; (4) rectal and vaginal administration such as suppositories, pessaries and foams; (5) inhalation and intranasal administration such as dry powders, aerosols, suspensions, and solutions (sprays and drops); (6) topical administration such as creams, ointments, lotions, solutions, pastes, drops, sprays, foams and gels; (7) ocular such as drops, ointment, sprays, suspensions and inserts; and (8) buccal and sublingual administration such as lozenges, patches, sprays, drops, chewing gums and tablets.

In the context of this invention, the term “pharmaceutically acceptable excipient” refers to any pharmaceutically acceptable material present in the dosage form other than 6-(3-cyclobutyl-2,3,4,5-tetrahydro-1H-benzo[d]azepin-7-yloxy)-N-methyl-nicotinamide or a pharmaceutically acceptable salt thereof and the stabiliser. Suitable pharmaceutically acceptable excipients will vary depending upon the particular dosage form chosen and include diluents, binders, disintegrants and superdisintegrants, lubricants, glidants, granulating agents, coating agents, wetting agents, solvents, co-solvents, suspending agents, emulsifiers, sweeteners, flavouring agents, flavour-masking agents, colouring agents, anticaking agents, humectants, chelating agents, plasticizers, viscosity increasing agents, rate modifying agents, preservatives, surfactants. The skilled person will appreciate that certain pharmaceutically acceptable excipients may serve more than one function and may serve alternative functions depending on how much of the excipient is present in the formulation and what other ingredients are present in the formulation. Guidance on the selection of suitable pharmaceutically acceptable excipients is available from Remington's Pharmaceutical Sciences (Mack Publishing Company).

The dosage forms of the invention may be prepared using techniques and methods known to those skilled in the art. Some of the methods commonly used in the art are described in Remington's Pharmaceutical Sciences (Mack Publishing Company).

In one embodiment, the invention is directed to a solid oral dosage form such as a tablet or capsule comprising 6-(3-cyclobutyl-2,3,4,5-tetrahydro-1H-benzo[d]azepin-7-yloxy)-N-methyl-nicotinamide or a pharmaceutically acceptable salt thereof, a stabiliser and a diluent. Suitable diluents include saccharides (e.g. lactose, sucrose, dextrose), sugar alcohols (e.g. mannitol, sorbitol), starch (e.g. corn starch, potato starch and pregelatinized starch), cellulose and its derivatives (e.g. microcrystalline cellulose), calcium sulphate and dibasic calcium phosphate. The dosage form may further comprise other conventional excipients such as binders, disintegrants, lubricants and glidants. Suitable binders include starch (e.g. corn starch, potato starch and pregelatinized starch), gelatin, acacia, sodium alginate, alginic acid, tragacanth, guar gum, polyvinylpyrrolidone, and cellulose and its derivatives (e.g. ethylcellulose, methylcellulose, carboxymethylcellulose, hydroxypropylcellulose and hydroxypropylmethylcellulose). Suitable disintegrants include starches, cross-linked polyvinylpyrrolidone, sodium starch glucolate, croscarmellose, alginic acid and sodium carboxymethyl cellulose. Suitable lubricants include stearic acid, magnesium stearate and calcium stearate. Suitable glidants include talc or colloidal silicon dioxide. The oral solid dosage form may further comprise an outer coating which may have cosmetic or functional properties.

In a more particular aspect, the present invention provides a dosage form for oral administration comprising a carrier tablet, which carrier tablet is at least partially covered by a film comprising:
a) 6-(3-cyclobutyl-2,3,4,5-tetrahydro-1H-benzo[d]azepin-7-yloxy)-N-methyl-nicotinamide or a pharmaceutically acceptable salt thereof, and
b) a stabiliser that reduces degradation of 6-(3-cyclobutyl-2,3,4,5-tetrahydro-1H-benzo[d]azepin-7-yloxy)-N-methyl-nicotinamide in the dosage form, when compared to a dosage form lacking said stabiliser.

In the context of this application, the term “carrier tablet” refers to a tablet that is substantially free of 6-(3-cyclobutyl-2,3,4,5-tetrahydro-1H-benzo[d]azepin-7-yloxy)-N-methyl-nicotinamide or pharmaceutically acceptable salts thereof. Typically, the tablet does not contain any therapeutic agent, although embodiments in which the carrier tablet contains one or more therapeutic agents are encompassed by the invention.

The composition of the carrier tablet is not important provided that it is pharmaceutically acceptable. The carrier tablet must, however, be of an appropriate size and shape to function as a tablet for oral administration. Any type of tablet may be used, for example, those described in Remington, The Science and Practice of Pharmacy, 21^{st} Edition, 2005 (Ed. D. B. Troy). In one embodiment, the carrier tablet is formed by conventional compression technology and comprises up to 100% w/w diluent, or a mixture of diluents. Conventional diluents include saccharides (e.g. lactose, sucrose, dextrose), sugar alcohols (e.g. mannitol, sorbitol), starch (e.g. corn starch potato starch and pregelatinized starch), cellulose and its derivatives (e.g. microcrystalline cellulose), calcium sulphate and dibasic calcium phosphate. The tablet may further comprise up to 100% w/w binder, or a mixture of binders. Suitable binders include starch (e.g. corn starch, potato starch and pre-gelatinized starch), gelatin, acacia, sodium alginate, alginic acid, tragacanth, guar gum, polyvinylpyrrolidone, and cellulose and its derivatives (e.g. ethylcellulose, methylcellulose, carboxymethylcellulose, hydroxypropylcellulose and hydroxypropylmethylcellulose). Carrier tablets may also contain other conventional excipients such as lubricants (e.g. stearic acid, magnesium stearate and calcium stearate) and glidants (e.g. talc or colloidal silicon dioxide). In one embodiment, lubricants and glidants are each present in an amount up to 10% w/w, more particularly up to 5% w/w. In another embodiment, substrates formed by injection moulding such as moulded tablets or capsule shells may be used as carrier tablets. Suitable thermoplastic materials for injection moulding include hydroxypropylcellulose, ethylcellulose, methacrylates and polyvinyl acetate.

In one embodiment, the carrier tablet is a tablet comprising microcrystalline cellulose (e.g. Avicel PH-102), pregelatinized starch (e.g. Starch 1500) and magnesium stearate. In a more particular embodiment, the carrier tablet has the following composition:

| Excipient | % w/w |
|---|---|
| Microcrystalline Cellulose (e.g. Avicel PH-102) | 90 |
| Pregelatinized starch (e.g. Starch 1500) | 9 |
| Magnesium Stearate | 1 |

In an alternative embodiment, the carrier substrate may be formulated such that it disintegrates in the mouth when administered orally, a so called “orally disintegrating tablet” or “ODT” substrate. Alternatively, the carrier substrate may be formulated so as to disintegrate in water, a so called “fast-dissolve tablet” or “FDT” substrate.

The carrier tablet provides a substrate or support for the film. In one embodiment, the carrier tablets are coated to substantially prevent absorption of 6-(3-cyclobutyl-2,3,4,5-tetrahydro-1H-benzo[d]azepin-7-yloxy)-N-methyl-nicotinamide or a pharmaceutically acceptable salt thereof by the carrier tablets. However, embodiments in which there is absorption of the 6-(3-cyclobutyl-2,3,4,5-tetrahydro-1H-benzo[d]azepin-7-yloxy)-N-methyl-nicotinamide or a pharmaceutically acceptable salt thereof by the carrier tablets are encompassed by the invention.

Suitable coatings for carrier tablets include aqueous film coats such as those commercially available from Colorcon, for example, the Opadry® coatings (“OPADRY WHITE 00F18484” or “OPADRY WHITE YS-1-7003”). Other suitable coatings include Surelease® (ethylcellulose). The dosage form may alternatively be coated with a film of gastroresistant and enterosoluble polymeric material. Suitable polymeric materials include cellulose acetophthalate, cellulose acetopropionate, cellulose trimellitate and acrylic and methacrylic copolymers. Colourings can be added.

In one embodiment, the carrier tablet is coated with a film coat to a 2-6% weight gain.

It will be appreciated that, in embodiments where the carrier tablet is coated to substantially prevent absorption of 6-(3-cyclobutyl-2,3,4,5-tetrahydro-1H-benzo[d]azepin-7-yloxy)-N-methyl-nicotinamide or a pharmaceutically acceptable salt thereof, the film coat selected must not be soluble in the solvent used during the manufacturing process of the dosage form. For example, where an aqueous solvent system is used, an aqueous film coat (like an Opadry®) will immediately disintegrate and a coating not soluble in water (e.g. Surelease® or Eudragit®) is appropriate.

In one embodiment, the film containing 6-(3-cyclobutyl-2,3,4,5-tetrahydro-1H-benzo[d]azepin-7-yloxy)-N-methyl-nicotinamide or a pharmaceutically acceptable salt thereof only partially coats the carrier tablet. In a more particular embodiment, the carrier tablets are shaped to contain one or more recesses or depressions. In such embodiments, the film containing 6-(3-cyclobutyl-2,3,4,5-tetrahydro-1H-benzo[d]azepin-7-yloxy)-N-methyl-nicotinamide or a pharmaceutically acceptable salt thereof may be substantially present within the recess of the carrier tablet.

The dosage form and/or the film that at least partially covers the carrier tablet comprises 6-(3-cyclobutyl-2,3,4,5-tetrahydro-1H-benzo[d]azepin-7-yloxy)-N-methyl-nicotinamide (the “free base”) or a pharmaceutically acceptable salt thereof. In the context of this invention, reference to the free base or pharmaceutically acceptable salt encompasses solvates and hydrates of the free base or pharmaceutically acceptable salt.

6-(3-Cyclobutyl-2,3,4,5-tetrahydro-1H-benzo[d]azepin-7-yloxy)-N-methyl-nicotinamide may be prepared according to known procedures, such as those disclosed in WO2004/056369. The disclosure of WO2004/056369 is incorporated herein by reference.

Pharmaceutically acceptable acid addition salts of 6-(3-cyclobutyl-2,3,4,5-tetrahydro-1H-benzo[d]azepin-7-yloxy)-N-methyl-nicotinamide include hydrobromide, hydrochloride, sulfate, nitrate, phosphate, succinate, maleate, formate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salts. Such salts can be formed by reaction with the appropriate acid, optionally in a suitable solvent such as an organic solvent, to give the salt which can be isolated for example by crystallisation and filtration.

The dosage form and/or the film may contain the free base, a pharmaceutically acceptable salt (stoichiometric or non-stoichiometric), or any mixture of these. In one embodiment, the dosage form contains the hydrochloride salt. In one embodiment, the film contains the hydrochloride salt.

In one embodiment of the invention, the dosage form and/or the film contains between 1 μg and 1 mg 6-(3-cyclobutyl-2,3,4,5-tetrahydro-1H-benzo[d]azepin-7-yloxy)-N-methyl-nicotinamide when measured as the amount of base present (that is, excluding any amount of acid added to form salts). In a more particular embodiment, the dosage form and/or the film contains between 1 μg and 500 μg, more particularly between 2 μg and 250 μg 6-(3-cyclobutyl-2,3,4,5-tetrahydro-1H-benzo[d]azepin-7-yloxy)-N-methyl-nicotinamide, when measured as the amount of free base present.

The dosage form and/or the film additionally contains a pharmaceutically acceptable stabiliser that reduces degradation of 6-(3-cyclobutyl-2,3,4,5-tetrahydro-1H-benzo[d]azepin-7-yloxy)-N-methyl-nicotinamide (or a pharmaceutically acceptable salt thereof) in the dosage form containing the stabiliser when compared to a dosage form lacking the stabiliser. The degradation of 6-(3-cyclobutyl-2,3,4,5-tetrahydro-1H-benzo[d]azepin-7-yloxy)-N-methyl-nicotinamide (or a pharmaceutically acceptable salt thereof) in the dosage form can be analysed by measuring the total impurity/degradation product content of the dosage form using gradient HPLC, using the method described below. The skilled reader will appreciate that, with the exception of the presence or absence of stabiliser, the dosage forms should otherwise be comparable, and should have been stored under similar conditions for similar time periods.

In one embodiment, the mean total impurity/degradation product content calculated from at least 3 samples of the dosage form containing stabiliser stored for 1 month at 40° C., 75% relative humidity is at least 50% lower than the mean total impurity/degradation product content calculated from at least 3 samples of a comparable dosage form lacking said stabiliser that was stored under comparable conditions.

In another embodiment, the mean total impurity/degradation product content of at least 3 samples of the dosage form containing stabiliser when stored at 30° C., 65% relative humidity for a period of 3 months should not exceed 10%, more particularly 5%.

Certain pharmaceutically acceptable antioxidants may act as stabilisers in the context of the present invention. Pharmaceutically acceptable antioxidants include those described in The Handbook of Pharmaceutical Excipients, Third Edition, 2000 (Ed. A. H. Kibbe). In one embodiment, the stabiliser is selected from the group consisting of citric acid, malic acid, ascorbic acid and its salts such as ascorbyl palmitate, lactic acid, tartaric acid, HCl (pH 1-5), butylated hydroxyanisole and butylated hydroxytoluene. Combinations of stabilisers may also be used in the present invention.

In a more particular embodiment, the stabiliser is selected from citric acid, ascorbic acid, HCl (pH1-5), butylated hydroxyanisole and butylated hydroxytoluene. Even more particularly, the stabiliser is citric acid.

The stabiliser or stabilisers must be present in the dosage form and/or the film in a sufficient amount to reduce degradation of 6-(3-cyclobutyl-2,3,4,5-tetrahydro-1H-benzo[d]azepin-7-yloxy)-N-methyl-nicotinamide or a pharmaceutically acceptable salt thereof. In certain embodiments comprising citric acid, the molar ratio of citric acid to free base may typically be in the range 0.5:1 to 550:1.

The film may additionally contain a film former, such as hydroxypropylcellulose, hydroxypropylmethyl cellulose, hydroxyethylcellulose, carboxymethyl cellulose, polyvinyl alcohol, polyvinyl pyrrolidone, carrageenan (kappa iota or lambda), gelatin, polyethylene glycol, polyethylene oxide, pullulan or acrylic polymer (e.g. EUDRAGIT grades RL, RS, E, L, S, FS30D) or any combinations thereof. In one embodiment, the film former is hydroxypropylcellulose. It will be apparent to the one skilled in the art, that any film former included in the dosage form should be soluble in the solvent used during its production.

The film may additionally contain other excipients. For example, it has been found that certain solvent systems, such as aqueous systems, require addition of surfactants (e.g. polysorbates (20, 40, 80), Triton 100, sodium lauryl sulphate or tyloxopol) and/or antifoaming agents (polydimethylsiloxane or dimethicone). Therefore, in a further embodiment, the film additionally contains one or more surfactants and/or one or more antifoaming agents.

The dosage form may be further coated. Suitable coatings include those listed above as suitable for coating of the carrier tablet. In one embodiment, the dosage form is coated to 2-6% weight gain.

The dosage form may optionally be packaged in a low oxygen environment. This may be achieved by inclusion of an oxygen scavenger in the packaging of the dosage form. Suitable oxygen scavengers include PharmaKeep® KH or KD (commercially available from Sud Chemie) and StabilOx™ speciality oxygen scavenger (commercially available from Multisorb Technologies). Alternatively, the dosage forms can be packaged in bottles that are impermeable to oxygen. Aluminium-aluminium blisters may also be used to package the dosage forms in a low oxygen environment.

In another aspect, the invention provides a method for preparing the dosage form of the invention. The method comprises dispensing a solution or suspension of 6-(3-cyclobutyl-2,3,4,5-tetrahydro-1H-benzo[d]azepin-7-yloxy)-N-methyl-nicotinamide or a pharmaceutically acceptable salt thereof and stabiliser onto a carrier tablet. Any solvent may be used provided that the stabiliser and any other excipients present in the film are soluble in the solvent. The solvent is typically volatile, and must be pharmaceutically acceptable in the (residual) quantities in which it appears in the finished dosage form.

Suitable solvents include water, organic solvents, propellants, liquefied gases and volatile silicones. In one embodiment, the solution or suspension of 6-(3-cyclobutyl-2,3,4,5-tetrahydro-1H-benzo[d]azepin-7-yloxy)-N-methyl-nicotinamide or a pharmaceutically acceptable salt thereof and stabiliser is prepared using an organic solvent, such as methanol, ethanol, acetone, acetic acid or methylene chloride. Mixtures of solvents (e.g. water-ethanol) may also be used. In one embodiment, the solvent is methanol.

In a further aspect, the invention provides a solution or suspension of 6-(3-cyclobutyl-2,3,4,5-tetrahydro-1H-benzo[d]azepin-7-yloxy)-N-methyl-nicotinamide or a pharmaceutically acceptable salt thereof and stabiliser in a solvent system. In one embodiment, the solution or suspension further comprises one or more film formers and/or surfactants and/or antifoaming agents. In another embodiment, the solvent is an organic solvent, such as methanol, ethanol, acetone, acetic acid or methylene chloride, more particularly methanol.

In certain embodiments in which the stabiliser is citric acid, it is present in the solution or suspension in an amount between 0.5-12% w/v. At very high concentrations of citric acid, the resulting dosage forms are “sticky” and may need to be film coated. In a more particular embodiment in which the stabiliser is citric acid, it is present in the solution or suspension in an amount between 0.5-6% w/v, particularly 2-3% w/v, more particularly 3% w/v.

In certain embodiments in which the stabiliser is butylated hydroxyanisole, it is present in the solution or suspension in an amount between 0.01-0.1% w/v.

In certain embodiments in which the stabiliser is butylated hydroxytoluene, it is present in the solution or suspension in an amount between 0.01-0.1% w/v.

In certain embodiments in which the stabiliser is ascorbic acid, it is present in the solution or suspension at 0.09% w/v.

In certain embodiments in which the film former is hydroxypropylcellulose, it is present in the solution or suspension in an amount between 4-6% w/v, particularly 5% w/v.

The carrier tablet and dispensed solution/suspension may be heated (e.g. in a forced air oven) to evaporate excessive liquid and result in the formation of a film upon at least a part of the surface of the carrier tablet. The dosage form may then optionally be film coated according to methods known in the art.

The carrier tablet used in the method for preparing the dosage form may have a recess or depression that provides a basin for the solution or suspension of 6-(3-cyclobutyl-2,3,4,5-tetrahydro-1H-benzo[d]azepin-7-yloxy)-N-methyl-nicotinamide or a pharmaceutically acceptable salt thereof and stabiliser to land after being dispensed. Typically, biconcave tablets having recesses on two faces of the tablet are employed. The two recesses can be used to receive the solution or suspension of 6-(3-cyclobutyl-2,3,4,5-tetrahydro-1H-benzo[d]azepin-7-yloxy)-N-methyl-nicotinamide or a pharmaceutically acceptable salt thereof and stabiliser. Alternatively, one of the recesses can be used to receive a solution or suspension of 6-(3-cyclobutyl-2,3,4,5-tetrahydro-1H-benzo[d]azepin-7-yloxy)-N-methyl-nicotinamide or a pharmaceutically acceptable salt thereof and stabiliser, and the remaining recess can be used to receive a solution or suspension of another therapeutic agent to produce a dosage form containing two different therapeutic agents. In a further embodiment, solutions of different therapeutic agents may be layered one on top of the other.

The dosage form of the present invention may be produced using the apparatus described in WO2005/123569 which publication is herein incorporated in its entirety. More particularly, the dosage form of the present invention may be produced by an apparatus containing a dispensing module for accurately dispensing a predetermined amount of the solution or suspension of 6-(3-cyclobutyl-2,3,4,5-tetrahydro-1H-benzo[d]azepin-7-yloxy)-N-methyl-nicotinamide or a pharmaceutically acceptable salt thereof and stabiliser onto the carrier tablets. The apparatus may also have a holding member for holding the carrier tablets, which may move continually along the apparatus as the dispensing module dispenses the solution/suspension onto each of the carrier tablets.

The apparatus may also have a drying system that dries or evaporates solvent from the solution/suspension deposited on each of the carrier tablets. The holding member may move continually along the apparatus as the drying system dries the dosage on each of the carrier tablets. The drying system may dry the dosage by use of heated air, infrared or microwave heating.

The apparatus may also have a coating system that applies a coating over the dosage form. The coating system may have a pad printing device or a sprayer that applies the coating to each of the carrier substrates. The holding member may move continually along the apparatus as the coating system applies the coating to each of the carrier tablets. The apparatus may also have a coating dryer that dries the coating on each of the carrier tablets.

It will be apparent that the apparatus described above could re-process carrier tablets any number of times in order to add solutions or suspensions of different therapeutic agents. Alternatively, the apparatus could have additional dispensing systems in series to add each of the solutions/suspensions to the carrier tablets.

6-(3-Cyclobutyl-2,3,4,5-tetrahydro-1H-benzo[d]azepin-7-yloxy)-N-methyl-nicotinamide and its pharmaceutically acceptable salts are H3 antagonists having useful therapeutic properties. More particularly, 6-(3-cyclobutyl-2,3,4,5-tetrahydro-1H-benzo[d]azepin-7-yloxy)-N-methyl-nicotinamide and its pharmaceutically acceptable salts are believed to be of potential use in the treatment of neurological diseases including Alzheimer's disease, dementia (including Lewy body dementia and vascular dementia), age-related memory dysfunction, mild cognitive impairment, cognitive deficit, epilepsy, migraine, Parkinson's disease, multiple sclerosis (including fatigue), stroke, pain of neuropathic origin (including neuralgias, neuritis and back pain), inflammatory pain (including osteoarthritis, rheumatoid arthritis, acute inflammatory pain and back pain) and sleep disorders (including hypersomnolence, excessive daytime sleepiness, narcolepsy, sleep deficits associated with Parkinson's disease and fatigue, especially in multiple sclerosis); psychiatric disorders including psychotic disorders (such as schizophrenia (particularly cognitive deficit of schizophrenia) and bipolar disorder), attention deficit hypereactivity disorder, depression (including major depressive disorder), anxiety and addiction; and other diseases including obesity and gastro-intestinal disorders.

Accordingly, in a further aspect, the present invention provides the dosage form for use in therapy. More particularly, the invention provides the dosage form for use in the treatment or prophylaxis of the above disorders, in particular neurological and psychiatric disorders.

The invention further provides a method of treatment or prophylaxis of the above disorders, in mammals including humans, which comprises administering to the sufferer the dosage form of the present invention.

In another aspect, the invention provides the use of 6-(3-cyclobutyl-2,3,4,5-tetrahydro-1H-benzo[d]azepin-7-yloxy)-N-methyl-nicotinamide or a pharmaceutically acceptable salt thereof in the manufacture of the dosage form of the invention for use in the treatment of the above disorders.

6-(3-Cyclobutyl-2,3,4,5-tetrahydro-1H-benzo[d]azepin-7-yloxy)-N-methyl-nicotinamide or a pharmaceutically acceptable salt thereof may be used in combination with other therapeutic agents. When 6-(3-cyclobutyl-2,3,4,5-tetrahydro-1H-benzo[d]azepin-7-yloxy)-N-methyl-nicotinamide or a pharmaceutically acceptable salt thereof is intended for use in the treatment of Alzheimer's disease, it may be used in combination with medicaments claimed to be useful as either disease modifying or symptomatic treatments of Alzheimer's disease. Suitable examples of such other therapeutic agents may be symptomatic agents, for example those known to modify cholinergic transmission such as M1 muscarinic receptor agonists or allosteric modulators, M2 muscarinic antagonists, acetylcholinesterase inhibitors (such as tetrahydroaminoacridine, donepezil hydrochloride and rivastigmine), nicotinic receptor agonists or allosteric modulators (such as α7 agonists or allosteric modulators or α4β2 agonists or allosteric modulators), PPAR agonists (such as PPARγ agonists), 5-HT₄ receptor partial agonists, 5-HT₆ receptor antagonists or 5HT1A receptor antagonists and NMDA receptor antagonists or modulators, or disease modifying agents such as β or γ-secretase inhibitors.

When 6-(3-cyclobutyl-2,3,4,5-tetrahydro-1H-benzo[d]azepin-7-yloxy)-N-methyl-nicotinamide or a pharmaceutically acceptable salt thereof is intended for use in the treatment of narcolepsy, it may be used in combination with medicaments claimed to be useful as treatments for narcolepsy. Suitable examples of such other therapeutic agents include modafinil, armodafinil and monoamine uptake blockers.

When 6-(3-cyclobutyl-2,3,4,5-tetrahydro-1H-benzo[d]azepin-7-yloxy)-N-methyl-nicotinamide or a pharmaceutically acceptable salt thereof is intended for use in the treatment of schizophrenia, it may be used in combination with medicaments claimed to be useful as treatments of schizophrenia including i) antipsychotics including typical antipsychotics (for example chlorpromazine, thioridazine, mesoridazine, fluphenazine, perphenazine, prochlorperazine, trifluoperazine, thiothixine, haloperidol, molindone and loxapine), atypical antipsychotics (for example clozapine, olanzapine, risperidone, quetiapine, aripirazole, ziprasidone, amisulpride and aripiprazole), glycine transporter 1 inhibitors and metabotropic receptor ligands; ii) drugs for extrapyramidal side effects, for example anticholinergics (such as benztropine, biperiden, procyclidine, and trihexyphenidyl) and dopaminergics (such as amantadine); iii) antidepressants including serotonin reuptake inhibitors (such as citalopram, escitalopram, fluoxetine, paroxetine, dapoxetine and sertraline), dual serotonin/noradrenaline reuptake inhibitors (such as venlafaxine, duloxetine and milnacipran), noradrenaline reuptake inhibitors (such as reboxetine), tricyclic antidepressants (such as amitriptyline, clomipramine, imipramine, maprotiline, nortriptyline and trimipramine), monoamine oxidase inhibitors (such as isocarboxazide, moclobemide, phenelzine and tranylcypromine), and others (such as buproprion, mianserin, mirtazepine, nefazodone and trazodone); iv) anxiolytics including benzodiazepines such as alprazolam and lorazepam; and v) cognitive enhancers for example cholinesterase inhibitors (such as tacrine, donepezil, rivastigmine and galantamine).

The invention thus provides, in a further aspect, a dosage form comprising a carrier tablet which carrier tablet is at least partially coated by a film comprising 6-(3-cyclobutyl-2,3,4,5-tetrahydro-1H-benzo[d]azepin-7-yloxy)-N-methyl-nicotinamide or a pharmaceutically acceptable salt thereof and a stabiliser, which dosage form further comprises an additional therapeutic agent or agents.

It will be apparent that the additional therapeutic agent may be present in the carrier tablet. Alternatively, as discussed above, a film containing additional therapeutic agents may be deposited on the carrier tablet. Where the carrier tablet has two recesses, one recess may contain the film containing 6-(3-cyclobutyl-2,3,4,5-tetrahydro-1H-benzo[d]azepin-7-yloxy)-N-methyl-nicotinamide or a pharmaceutically acceptable salt thereof and a stabiliser, and the second recess may contain the film containing the additional therapeutic agent or agents. Alternatively, the films containing 6-(3-cyclobutyl-2,3,4,5-tetrahydro-1H-benzo[d]azepin-7-yloxy)-N-methyl-nicotinamide or a pharmaceutically acceptable salt thereof and a stabiliser, and the additional therapeutic agent may be layered one on top of the other.

When 6-(3-cyclobutyl-2,3,4,5-tetrahydro-1H-benzo[d]azepin-7-yloxy)-N-methyl-nicotinamide or a pharmaceutically acceptable salt thereof is used in combination with a second therapeutic agent active against the same disease state the dose of 6-(3-cyclobutyl-2,3,4,5-tetrahydro-1H-benzo[d]azepin-7-yloxy)-N-methyl-nicotinamide or the pharmaceutically acceptable salt thereof may differ from that when the 6-(3-cyclobutyl-2,3,4,5-tetrahydro-1H-benzo[d]azepin-7-yloxy)-N-methyl-nicotinamide or the pharmaceutically acceptable salt thereof is formulated alone. Appropriate doses will be readily appreciated by those skilled in the art.

The following examples illustrate the invention but do not limit it in any way.

### EXAMPLES

### Example 1

### Preparation of 6-(3-Cyclobutyl-2,3,4,5-tetrahydro-1H-benzo[d]azepin-7-yloxy)-N-methyl-nicotinamide hydrochloride

6-(3-Cyclobutyl-2,3,4,5-tetrahydro-1H-benzo[d]azepin-7-yloxy)-N-methyl-nicotinamide (4.0 kg) and methanol (7.9 kg) are charged to a reactor with stirring at 20 to 25° C. for at least 30 minutes. Acetyl chloride (1.0 kg) is added in methanol (7.9 kg) at 0 to 10° C. and then slowly charged into the above reaction solution at 15 to 25° C. The reaction is then stirred at 45 to 55° C. until reaction mixture becomes brown clear solution. The reaction solution is filtered hot and then vacuum concentrated at not more than 50° C. to about ½ volume to afford white slurry mixture. Ethyl acetate (18.0 kg) is slowly charged into the reaction solution at 45 to 55° C. and then stirred at this temperature for at least 1 hour. The white slurry mixture is cooled to 15 to 25° C. at ramping rate 10° C./hr and then stirred at this temperature for not less than 24 hours. The slurry mixture is filtered, and the cake is washed by Ethyl acetate (3.6 Kg×2) twice to give the “wet cake” 6-(3-cyclobutyl-2,3,4,5-tetrahydro-1H-benzo[d]azepin-7-yloxy)-N-methyl-nicotinamide hydrochloride as a white solid (6.203 Kg).

The intermediate grade 6-(3-cyclobutyl-2,3,4,5-tetrahydro-1H-benzo[d]azepin-7-yloxy)-N-methyl-nicotinamide hydrochloride (6.203 Kg) and methanol (7.2 Kg) are charged into a reactor and then heated to 45 to 55° C. Ethyl acetate (17.7 Kg) is slowly charged at 45 to 55° C., and then the solution is stirred at this temperature for at least 1 hour. The white slurry mixture is cool to 15-25° C. for at ramping rate 10° C./hr and then stirred at this temperature for not less than 24 hours. The slurry mixture is filtered, and the wet cake is washed with ethyl acetate (3.6 Kg×2). The product is vacuum dried at not more than 50° C. to give 6-(3-cyclobutyl-2,3,4,5-tetrahydro-1H-benzo[d]azepin-7-yloxy)-N-methyl-nicotinamide hydrochloride as a white solid (3.034 Kg, 77% th).

### Example 2

### Preparation of round tablets containing 0.002 mg 6-(3-cyclobutyl-2,3,4,5-tetrahydro-1H-benzo[d]azepin-7-yloxy)-N-methyl-nicotinamide

The core components were passed through a nominal 30 mesh screen and then blended together in a suitable blender and compressed on a rotary tablet press to produce round biconcave tablets with a diameter of 7.94 mm. Compression was followed by de-dusting and metal checking. The tablets were then transferred to a coating pan and coated to a target 4% (w/w) gain.

The composition of the carrier tablets is given below:

| | |
|---|---|
| Component | Weight (mg) |

Core

| | |
|---|---|
| Microcrystalline cellulose (Avicel PH-102) | 158.4 |
| Pregelatinized Starch (Starch 1500) | 15.84 |
| Magnesium Stearate | 1.76 |
| Total | 176 |

Film Coat

| | |
|---|---|
| Opadry White 00F18484 | 7.04 |
| Purified Water | qs* |
| Total (core + film coat) | 183 |

*Removed during processing

6-(3-Cyclobutyl-2,3,4,5-tetrahydro-1H-benzo[d]azepin-7-yloxy)-N-methyl-nicotinamide hydrochloride was dissolved in carrier solution (5% w/v hydroxypropylcellulose, 3% w/v citric acid in methanol) to a final concentration of 0.58 mg/g (w/w).

Approximately 3.9 mg dosing solution was dispensed onto each tablet in an array of carrier tablets. The tablets were dried in a forced air oven at −50° C.

### Example 3

### Preparation of round tablets containing 0.01 mg 6-(3-cyclobutyl-2,3,4,5-tetrahydro-1H-benzo[d]azepin-7-yloxy)-N-methyl-nicotinamide

Tablets containing 0.01 mg 6-(3-cyclobutyl-2,3,4,5-tetrahydro-1H-benzo[d]azepin-7-yloxy)-N-methyl-nicotinamide were prepared in the manner described in Example 2 except that the concentration of 6-(3-cyclobutyl-2,3,4,5-tetrahydro-1H-benzo[d]azepin-7-yloxy)-N-methyl-nicotinamide hydrochloride in the dosing solution was 2.83 mg/g (w/w) and the amount dispensed was approximately 4 mg.

### Examples 4-7

### Preparation of round tablets containing 0.002 mg, 0.01 mg, 0.05 mg and 0.25 mg 6-(3-cyclobutyl-2,3,4,5-tetrahydro-1H-benzo[d]azepin-7-yloxy)-N-methyl-nicotinamide

Tablets containing 0.002 mg, 0.01 mg, 0.05 mg and 0.25 mg 6-(3-cyclobutyl-2,3,4,5-tetrahydro-1H-benzo[d]azepin-7-yloxy)-N-methyl-nicotinamide were prepared in the manner described in Example 2 except that the carrier solution contained 2% w/v citric acid and the concentration of 6-(3-cyclobutyl-2,3,4,5-tetrahydro-1H-benzo[d]azepin-7-yloxy)-N-methyl-nicotinamide hydrochloride in the dosing solution and the amount of the dosing solution dispensed was varied as follows:

| Dose | Concentration w/w (mg/g) | Amount dispensed (mg) |
|---|---|---|
| 0.002 | 0.45 | 5.04 |
| 0.01 | 2.25 | 5.06 |
| 0.05 | 10.55 | 5.39 |
| 0.25 | 59.36 | 4.78 |

### Examples 8-11

### Preparation of round tablets containing 0.002 mg, 0.01 mg, 0.05 mg and 0.25 mg 6-(3-cyclobutyl-2,3,4,5-tetrahydro-1H-benzo[d]azepin-7-yloxy)-N-methyl-nicotinamide

Tablets containing 0.002 mg, 0.01 mg, 0.05 mg, and 0.25 mg 6-(3-cyclobutyl-2,3,4,5-tetrahydro-1H-benzo[d]azepin-7-yloxy)-N-methyl-nicotinamide were prepared in the manner described in Example 2 except that the carrier solution contained 12% w/v citric acid and 4% w/v hydroxypropylcellulose, and the concentration of 6-(3-cyclobutyl-2,3,4,5-tetrahydro-1H-benzo[d]azepin-7-yloxy)-N-methyl-nicotinamide hydrochloride in the dosing solution and the amount of the dosing solution dispensed was varied as follows:

| Dose | Concentration w/w (mg/g) | Amount dispensed (mg) |
|---|---|---|
| 0.002 | 0.57 | 3.97 |
| 0.01 | 2.86 | 3.96 |
| 0.05 | 14.32 | 3.97 |
| 0.25 | 71.43 | 3.98 |

The following examples (Example 12 and Example 13) are representative of an example tablet which may be prepared in accordance with the invention:

### Example 12

### Preparation of a Orally Disintegrating Tablet (ODT) Carrier Substrate

### a) Preparation of ODT Carrier Substrate

StarLac and Neotame are passed through a nominal 20 mesh screen. The mixture and unsieved mint flavoring are transferred to a suitable blender and blended for approximately 10 minutes. Magnesium stearate is passed through a nominal 30 mesh screen, transferred to the blender and the entire mixture blended for approximately 2 minutes. The weights of material used are calculated from the percentage weights given in Table A. The blend is compressed to meet the desired specifications (for example round, biconcave tablets, range in diameter from ˜8 mm to ˜9.5 mm) on a suitable rotary press utilizing appropriate tablet tooling. The tablets may be passed through a de-duster and metal checker.

TABLE A

| COMPOSITION | SPECIFICATION | % (w/w) | FUNCTION |
|---|---|---|---|
| StarLac* | Non Compendial | 98.5% | Diluent |
| Mint Flavoring | Non Compendial | 0.9% | Flavoring |
| Neotame | NF | 0.1% | Sweetener |
| Magnesium Stearate | Ph. Eur/USP-NF/JP | 0.5% | Lubricant |

*StarLac: mixture of 85% alpha-lactose monohydrate (Ph. Eur./USP-NF) and 15% maize starch (Ph. Eur./USP-NF)

### b) Preparation of Ethylcellulose Coat for Application to Tablet by Pad-Printing

Ethylcellulose is dissolved in methanol with stirring, and triethyl citrate added. The weights of material used are calculated from the percentage weights given in Table B. Sufficient methanol is added to bring to target on w/w basis. The solution is transferred to the ink cup of a pad printing machine equipped with a suitable image cliché with a round image, slightly smaller diameter then the actual tablet diameter. A suitable polymer pad is installed to match the cliché image plate. Tablets are presented to the pad printer in a defined array, matching the cliché. The pad printer may apply 2-4 tamps to the carrier tablet to apply a coat that will provide a protective layer to mitigate solvent infiltration into the uncoated carrier substrate during the liquid dispensing process.

TABLE B

| COMPOSITION | SPECIFICATION | % (w/w) | FUNCTION |
|---|---|---|---|
| Ethylcellulose | NF | 30 | Protective |
| | | | barrier coat |
| Triethyl Citrate | Ph. Eur./USP-NF | 1.67 | Plasticiser |
| Methanol | Ph. Eur./USP-NF | qs*** to 100 | vehicle |

***Methanol eliminated by evaporation.

### Example 13

### Alternative preparation of an Orally Disintegrating Tablet (ODT) Carrier Substrate

Mannitol, crospovidone XL, xylitol and Neotame are passed through a nominal 20 mesh screen, the mixture and the unsieved Mint Flavoring transferred to a suitable blender and blended for approximately 10 minutes. Magnesium stearate and colloidal silicon dioxide are passed through a nominal 30 mesh screen, transferred to the blender and the entire mixture blended for approximately 2 minutes. The weights of material used are calculated from the percentage weights given in Table C. The blend is compressed to meet the desired specifications (for example round, biconcave tablets, range in diameter from ˜8 mm to ˜9.5 mm) on a suitable rotary press utilizing an appropriate tablet tooling. The tablets are passed through a de-duster and metal checker.

An ethylcellulose coat may be prepared and applied as described for Example 12.

TABLE C

| COMPOSITION | SPECIFICATION | % (w/w) | FUNCTION |
|---|---|---|---|
| Mannitol (Grade 300 | Ph. Eur/USP-NF/JP | 73.15% | Diluent/ |
| Direct Compression) | | | sweetener |
| Crospovidone XL | Ph. Eur/USP-NF | 20.00% | Disintegrant |
| Xylitol (Grade 300 | Ph. Eur/USP-NF/JP | 5.00% | Diluent/ |
| for Direct | | | sweetener |
| Compression) | | | |
| Mint Flavoring | Non Compendial | 0.90% | Flavoring |
| Neotame | NF | 0.10% | Sweetener |
| Magnesium Stearate | Ph. Eur/USP-NF/JP | 0.75% | Lubricant |
| Colloidal silicon | Ph. Eur/USP-NF/JP | 0.10% | Lubricant |
| dioxide | | | |

PROPERTIES: The stability of the drug substance in the tablets may be tested as set out below:

Dissolve tablets 5-10 tablets in diluent (0.1M hydrochloric acid) to produce a final concentration of active agent of 0.025 mg/ml and sonicate for approximately 15 minutes with occasional shaking. Centrifuge an aliquot of the sample at 10,000 rpm for 5 mins (for low dose tablets, a two step centrifugation in recommended, centrifuging first at 3,500 rpm for 15 mins, and then centrifuging the supernatant at 10,000 rpm for 5 mins) to give a clear solution for injection on the HLPC. Prepare samples using placebo tablets to act as control samples.

Using the following instrument conditions, equilibrate the chromatographic system with A. Record the chromatograms for the sample and placebo preparations.

Column: Phenomenex Luna C18(2), 3 μm, 150×4.6 mm.

Column Temperature: 40° C.

Mobile Phase A: water+0.05% (v/v) trifluoroacetic acid

Mobile Phase B: acetonitrile+0.05% (v/v) trifluoroacetic acid

Flow Rate: 1.0 ml/min

Detector wavelength: 240 nm

Injection volume: 100 μl

Gradient Profile:

| Time (min) | % A | % B |
|---|---|---|
| 0 | 100 | 0 |
| 25 | 70 | 30 |
| 40 | 0 | 100 |
| 40.1 | 100 | 0 |
| 50 | 100 | 0 |

Following comparison of the chromatograms to identify impurities and degradation products, the percentage content of each impurity/degradation product in the control and sample injections can be calculated by dividing the area of the impurity/degradation product peak by the summed total of the peak for 6-(3-cyclobutyl-2,3,4,5-tetrahydro-1H-benzo[d]azepin-7-yloxy)-N-methyl-nicotinamide and all impurities/degradation products, and multiplying by 100.

The total impurity content can be calculated by summing the percentage content of each impurity/degradation product present. Typically, only impurities/degradation products present in an amount of greater than or equal to 0.05 or 0.03% are included in the calculation of total impurity/degradation product content.

1. A dosage form comprising:
a) 6-(3-cyclobutyl-2,3,4,5-tetrahydro-1H-benzo[d]azepin-7-yloxy)-N-methyl-nicotinamide or a pharmaceutically acceptable salt thereof;
b) a stabiliser, which reduces degradation of 6-(3-cyclobutyl-2,3,4,5-tetrahydro-1H-benzo[d]azepin-7-yloxy)-N-methyl-nicotinamide in the dosage form when compared to a dosage form lacking said stabiliser; and
c) a pharmaceutically acceptable excipient.

2. A dosage form according to claim 1 which comprises a carrier tablet, which carrier tablet is at least partially covered by a film comprising:
a) 6-(3-cyclobutyl-2,3,4,5-tetrahydro-1H-benzo[d]azepin-7-yloxy)-N-methyl-nicotinamide or a pharmaceutically acceptable salt thereof, and
b) a stabiliser that reduces degradation of 6-(3-cyclobutyl-2,3,4,5-tetrahydro-1H-benzo[d]azepin-7-yloxy)-N-methyl-nicotinamide in the dosage form, when compared to a dosage form lacking said stabiliser.

3. A dosage form according to claim 1, which contains between 1 μg and 1 mg of 6-(3-cyclobutyl-2,3,4,5-tetrahydro-1H-benzo[d]azepin-7-yloxy)-N-methyl-nicotinamide, when measured as the amount of free base present.

4. A dosage form according to claim 1, which comprises 6-(3-cyclobutyl-2,3,4,5-tetrahydro-1H-benzo[d]azepin-7-yloxy)-N-methyl-nicotinamide hydrochloride.

5. A dosage form according to claim 1, wherein said stabiliser is selected from the group consisting of citric acid, malic acid, ascorbic acid and its salts, butylated hydroxyanisole and butylated hydroxytoluene.

6. A dosage form according to claim 2, wherein said stabiliser is citric acid, and wherein the molar ratio of citric acid to free base is in the range 0.5:1 to 550:1.

7. A dosage form according to claim 1, wherein said carrier tablet has at least one recess.

8. A dosage form according to claim 7, wherein said film is present in a recess on said carrier tablet.

9. A dosage form according to claim 1, wherein there is substantially no absorption of 6-(3-cyclobutyl-2,3,4,5-tetrahydro-1H-benzo[d]azepin-7-yloxy)-N-methyl-nicotinamide or a pharmaceutically acceptable salt thereof by the carrier tablet.

10. A dosage form according to claim 9, wherein said carrier tablet is coated.

11. A dosage form according to claim 1, wherein said dosage form is further coated.

12-13. (canceled)

14. A method of producing a dosage form as defined in claim 2, comprising dispensing a solution or suspension of 6-(3-cyclobutyl-2,3,4,5-tetrahydro-1H-benzo[d]azepin-7-yloxy)-N-methyl-nicotinamide or a pharmaceutically acceptable salt thereof and stabiliser onto a carrier tablet.

15. A method of treatment of neurological diseases which comprises administering to a host in need thereof a dosage form as defined in claim 1.

16-17. (canceled)

18. A solution or suspension of 6-(3-cyclobutyl-2,3,4,5-tetrahydro-1H-benzo[d]azepin-7-yloxy)-N-methyl-nicotinamide or a pharmaceutically acceptable salt thereof and stabiliser in a solvent system.
